(19) United States
(12) Patent Application Publication
(10) Pub. No.: US 2014/0350240 A1
(43) Pub. Date: Nov. 27, 2014

(54) PROCESS FOR PREPARING CEFTAROLINE SALTS OR HYDRATES THEREOF

(71) Applicant: RANBAXY LABORATORIES LIMITED, New Delhi, Delhi (IN)

(72) Inventors: Kallimulla Mohammad, Krishna (IN); Bishwa Prakash Rai, Azamgarh (IN); Neera Tewari, Gurgaon (IN)

(21) Appl. No.: 14/363,353

(22) PCT Filed: Dec. 5, 2012
(86) PCT No.: PCT/IB2012/057002

§ 371 Date: Jun. 6, 2014

(30) Foreign Application Priority Data
Dec. 7, 2011 (IN) 3562/DEL/2011

(51) Int. Cl. C07D 501/06

(52) U.S. Cl. CPC C07D 501/06
USPC 540/225

(57) ABSTRACT

The present invention relates to a process for the preparation of ceftaroline salts or hydrates thereof.

### FIELD OF THE INVENTION

The present invention relates to a process for the preparation of ceftaroline salts or hydrates thereof.

### BACKGROUND OF THE INVENTION

Ceftaroline fosamil is chemically known as (6,7)-7-{(2Z)-2-(ethoxyimino)-2-[5-(phosphonoamino)-1,2,4-thiadiazol-3-yl]acetamido}-3-{[4-(1-methyl pyridin-1-ium-4-yl)-1,3-thiazol-2-yl]sulfanyl}-8-oxo-5-thia-1-azabicyclo[4.2.0]oct-2-ene-2-carboxylate monoacetate monohydrate, of Formula I.

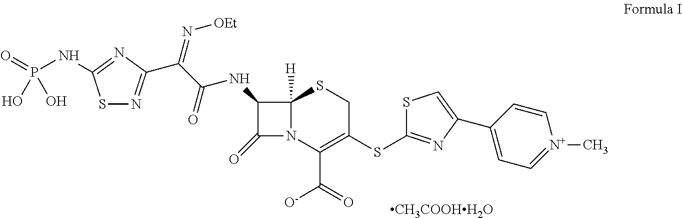

Ceftaroline fosamil is a phosphate prodrug of ceftaroline of Formula II and is indicated for the treatment of acute bacterial skin and skin structure infections and community-acquired bacterial pneumonia.

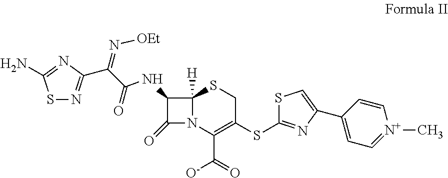 [SMILES: CCO/N=C(\C(=O)N[C@@H]1C(=O)N2C(C(=O)[O-])=C(Sc3nc(-c4cc[n+](C)cc4)cs3)CS[C@H]12)c1nsc(N)n1]

Japan Patent No. 3927262 B2 provides a process for the preparation of ceftaroline, wherein 7-amino-3-[4-(1-methylpyridinium-4-yl)thiazol-2-yl]thio-3-cephem-4-carboxylate is dissolved in a water-tetrahydrofuran mixture (1:2) in the presence of aqueous sodium bicarbonate. This solution is treated with 2-(5-amino-1,2,4-thiadiazol-3-yl)-2(Z)-ethoxyiminoacetyl chloride hydrochloride, followed by distillation, to obtain a residue. The residue is subjected to Diaion HP-20 chromatography and lyophilized to provide lyophilized ceftaroline.

U.S. Pat. No. 6,417,175 provides a process for the preparation of ceftaroline fosamil, wherein 7β-amino-3-[4-(1-methyl-4-pyridinio)-2-thiazolylthio]-3-cephem-4-carboxylate hydrochloride is treated with 2-(5-dichlorophosphorylamino-1,2,4-thiadiazol-3-yl)-2(Z)-ethoxyiminoacetyl chloride to obtain a mixture containing ceftaroline fosamil.

The mixture is purified by column chromatography and the fractions containing the desired compound are concentrated under reduced pressure. The concentrate is lyophilized to obtain a lyophilized ceftaroline fosamil.

The steps of chromatographic elution and lyophilization make the processes of Japan Patent No. 3927262 B2 and U.S. Pat. No. 6,417,175 unsuitable at an industrial scale. Further, Japan Patent No. 3927262 B2 and U.S. Pat. No. 6,417,175 do not provide a method of preparing ceftaroline salts.

Japan Patent No. 4656798 B2 provides a process for the preparation of crystalline ceftaroline hydrochloride or hydrates thereof, wherein ceftaroline fosamil or its acetic acid solvate is treated with hydrochloric acid to provide ceftaroline hydrochloride. Further, the process provided in the Japan Patent No. 4656798 B2 does not disclose purity of ceftaroline hydrochloride.

The Japan Patent No. 4656798 B2 discloses that ceftaroline has stability and quality control problems. Ceftaroline salts or hydrates thereof, for example, ceftaroline dihydrochloride of Formula IIa, have superior stability to ceftaroline.

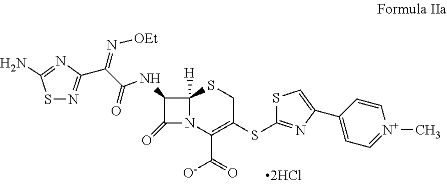 [SMILES: CCO/N=C(\C(=O)N[C@@H]1C(=O)N2C(C(=O)[O-])=C(Sc3nc(-c4cc[n+](C)cc4)cs3)CS[C@H]12)c1nsc(N)n1]

Japan Patent No. 4656798 B2 provides a method for the preparation of ceftaroline hydrochloride or hydrates thereof, wherein either ceftaroline fosamil or its acetic acid solvate is used as a starting material. The preparation of ceftaroline fosamil or its acetic acid solvate, followed by its conversion into ceftaroline hydrochloride or hydrates thereof, increases the number of steps and makes the process costly. Therefore, there is a need to develop a simple, cost effective, and industrially feasible process for the preparation of ceftaroline salts or hydrates thereof.

### SUMMARY OF THE INVENTION

The present invention relates to a process for the preparation of ceftaroline salts or hydrates thereof.

An aspect of the present invention relates to a process for the preparation of a ceftaroline salt or a hydrate thereof, wherein the process comprises the steps of:

(a) treating a compound of Formula III

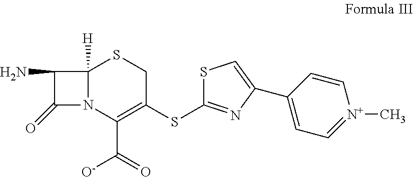 [SMILES: C[n+]1ccc(-c2csc(SC3=C(C(=O)[O-])N4C(=O)[C@@H](N)[C@H]4SC3)n2)cc1]

or an ester derivative or a salt thereof with a compound of Formula IV

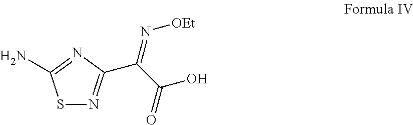 [SMILES: CCO/N=C(\C(=O)O)c1nsc(N)n1]

or a salt or a reactive derivative thereof to obtain a reaction mass; and

(b) treating the reaction mass with a salt-forming agent to obtain the ceftaroline salt or a hydrate thereof.

### BRIEF DESCRIPTION OF THE DRAWINGS

FIG. 1 depicts the XRPD (X-Ray Powder Diffractogram) of ceftaroline dihydrochloride obtained according to Example 2.

### DETAILED DESCRIPTION OF THE INVENTION

The present invention relates to a process for the preparation of ceftaroline salts or hydrates thereof. The present inventors have found that ceftaroline salts or hydrates thereof can be directly obtained without the isolation of ceftaroline. The process described herein also avoids the preparation of ceftaroline fosamil or its acetic acid solvate for the preparation of ceftaroline salts. Accordingly, the number of steps involved in the preparation of ceftaroline salts or a hydrate thereof is reduced. The single-step process developed by the present inventors is simple, cost effective, industrially feasible, and provides ceftaroline salts or a hydrate thereof in high purity.

An aspect of the present invention relates to a process for the preparation of a ceftaroline salt or hydrates thereof, wherein the process comprises the steps of:

(a) treating a compound of Formula III

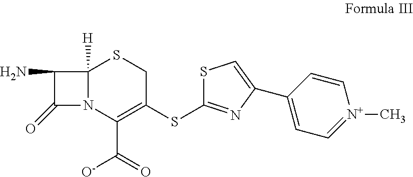 [SMILES: C[n+]1ccc(-c2csc(SC3=C(C(=O)[O-])N4C(=O)[C@@H](N)[C@H]4SC3)n2)cc1]

or an ester derivative or a salt thereof with a compound of Formula IV

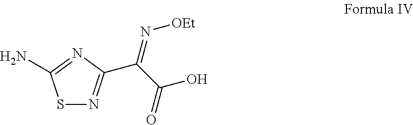 [SMILES: CCO/N=C(\C(=O)O)c1nsc(N)n1]

or a salt or a reactive derivative thereof to obtain a reaction mass; and

(b) treating the reaction mass with a salt-forming agent to obtain a ceftaroline salt or hydrates thereof.

The compound of Formula III or an ester derivative or salt thereof and the compound of Formula IV or a salt or reactive derivative thereof can be prepared by any method provided in the prior art, for example, Japan Patent No. 3927262 B2 or U.S. Pat. No. 6,906,055, or as described herein. The compound of Formula III or an ester derivative or salt thereof and the compound of Formula IV or a salt or reactive derivative thereof are treated in a solvent. When a reactive derivative of the compound of Formula IV is used, it may be reacted with the compound of Formula III after isolation from the reaction mixture in which it is formed, or the reaction mixture containing the reactive derivative of the compound of Formula IV can also be used for the reaction with the compound of Formula III. When the compound of Formula IV is used in the form of a free acid or a salt, a condensing agent, for example, N,N-dicyclohexylcarbodiimide, may be used. The compound of Formula III and the compound of Formula IV may be treated at about 10° C. to about 50° C. The compound of Formula III and the compound of Formula IV are treated in a solvent which does not interfere with the reaction. The solvent may be, for example, dioxane, tetrahydrofuran, dimethylformamide, dimethylacetamide, acetone, acetonitrile, dimethylsulfoxide, water, or a mixture thereof. A base is added to the resulting reaction mixture. The base may be added as such or in the form of a solution. A base may be, for example, tributylamine, triethylamine, sodium carbonate, potassium carbonate, calcium carbonate, or a mixture thereof. The base may be added to the reaction mixture over about 5 minutes to about 20 minutes. The addition of base may be followed by stirring for about 3 hours to about 5 hours at about 10° C. to about 50° C. A water-immiscible solvent is added to the resulting mass. The water-immiscible solvent may be, for example, ethyl acetate, isopropyl acetate, n-propyl acetate, n-butyl acetate, isobutyl acetate, amyl acetate, methylene chloride, chloroform, carbon tetrachloride, amyl chloride, cyclohexane, methylcyclohexane, isopropyl ether, diethyl carbonate, methyl isobutyl ketone, diisopropyl ketone, diisobutyl ketone, cyclohexanone, benzene, toluene, xylene, or mixtures thereof. The aqueous layer is separated, and a water-miscible solvent and a salt-forming agent are added to the aqueous layer. The water-miscible solvent may be, for example, acetonitrile, methanol, ethanol, propanol, isopropanol, acetone, or a mixture thereof. When hydrochloric acid is used as a salt forming agent, the normality of hydrochloric acid may range from about 2N to about 12N. The salt-forming agent may be added as such or in the form of a solution. The water-miscible solvent and a salt-forming agent may be added at about 5° C. to about 30° C. The addition of a water-miscible solvent and a salt-forming agent may be followed by stirring at about 5° C. to about 30° C. for about 8 hours to about 15 hours. The solid so obtained may be isolated from the mixture by methods including concentration, distillation, decantation, filtration, evaporation, centrifugation, or a combination thereof and the solid may further be dried.

In an embodiment of the invention, the salt of the compound of Formula III may be treated with the reactive derivative of the compound of Formula IV.

The term “ceftaroline salt” in the present invention includes, for example, inorganic basic salt, ammonium salt, organic basic salt, inorganic acid salt, and organic acid salt. Examples of ceftaroline salts include sodium salt, potassium salt, calcium salt, ethanolamine salt, diethanolamine salt, N-methylglucosamine salt, hydrochloride salt, dihydrochloride salt, hydrobromide salt, sulfuric acid salt, nitric acid salt, phosphoric acid salt, p-toluenesulfonic acid salt, methanesulfonic acid salt, formic acid salt, trifluoroacetic acid salt, maleic acid salt, lysine salt, arginine salt, ornithine salt, and histidine salt.

The term “hydrates” in the present invention includes, for example, hemihydrate, monohydrate, sequihydrate, dihydrate, trihydrate, tetrahydrate, and pentahydrate.

The salt of the compound of Formula III in the present invention includes, for example, inorganic basic salt, ammonium salt, organic basic salt, inorganic acid salt, and organic acid salt. Examples of salt include sodium salt, potassium salt, calcium salt, trimethylamine salt, triethylamine salt, tert-butyldimethylamine salt, dibenzylmethylamine salt, benzyldimethylamine salt, N,N-dimethylaniline salt, pyridine salt, quinoline salt, hydrochloride salt, hydrobromide salt, sulfate salt, nitrate salt, phosphate salt, formate salt, acetate salt, trifluoroacetate salt, methanesulfonate salt, and p-toluenesulfonate salt.

The “ester derivative of the compound of Formula III” in the present invention means an ester producible by esterifying the carboxyl group in the molecule which may be utilized as an intermediate in the synthesis and is non-toxic. Examples of esters which may be utilized as intermediates in the synthesis include an optionally substituted C₁₋₆ alkyl ester, a C₃₋₁₀ cycloalkyl ester, a C₃₋₁₀ cycloalkyl-C₁₋₆ alkyl ester, an optionally substituted C₆₋₁₀ aryl ester, an optionally substituted C₇₋₁₂ aralkyl ester, a di-C₆₋₁₀ aryl-methyl ester, a tri-C₆₋₁₀ aryl-methyl ester, a substituted silyl ester, and a C₂₋₆ alkanoyloxy-C₁₋₆ alkyl ester.

The optionally substituted C₁₋₆ alkyl in the present invention includes, for example, methyl, ethyl, propyl, isopropyl, butyl, isobutyl, t-butyl, pentyl, 2,2-dimethyl propyl, and hexyl, which may optionally be substituted with one or more groups including, for example, benzyloxy, methyl sulfonyl, fluorine, chlorine, bromine, acetyl, dimethylamino, pyridyl, and cyano.

The C₃₋₁₀ cycloalkyl in the present invention includes, for example, cyclopropyl, cyclobutyl, cyclopentyl, cyclohexyl, cycloheptyl, cyclooctyl, and cyclodecyl.

The C₃-₁₀ cycloalkyl-C₁₋₆ alkyl in the present invention includes, for example, cyclopropylmethyl, cyclopentylmethyl, and cyclohexylmethyl.

The optionally substituted C₆₋₁₀ aryl in the present invention includes, for example, phenyl, naphthyl, and biphenylyl, which may optionally be substituted with one or more groups including, nitro, fluorine, chlorine, and bromine.

The optionally substituted C₇-₁₂ aralkyl in the present invention includes, for example, benzyl, 1-phenylethyl, 2-phenylethyl, phenylpropyl, and naphthylmethyl, which may optionally be substituted with one or more groups including nitro, methoxy, methyl, ethyl, and hydroxy.

The di-C₆₋₁₀ aryl-methyl in the present invention includes, for example, benzhydryl.

The tri-C₆₋₁₀ aryl-methyl in the present invention includes, for example, trityl.

The substituted silyl in the present invention includes, for example, trimethylsilyl, tert-butyldimethylsilyl, and —Si(CH₃)₂CH₂CH₂ Si(CH₃)₂—.

The C₂₋₆ alkanoyloxy-C₁₋₆ alkyl in the present invention includes, for example, acetoxymethyl.

The salt of the compound of Formula IV in the present invention includes an inorganic basic salt and an organic basic salt. Examples of salts include sodium salts, potassium salts, calcium salts, trimethylamine salts, triethylamine salts, tert-butyldimethylamine salts, dibenzylmethylamine salts, benzyldimethylamine salts, N,N-dimethylaniline salts, pyridine salts, and quinoline salts.

The reactive derivative of the compound of Formula IV in the present invention includes, for example, acid halides, acid azides, acid anhydrides, mixed acid anhydrides, active amides, active esters, and active thio esters. Examples of reactive derivatives include acid chloride, acid amides of a free acid, di-ethoxyphosphoric acid ester, p-nitrophenyl ester, cyanomethyl ester, pentachlorophenyl ester, N-hydroxysuccinimide ester, N-hydroxy phthalimide ester, 1-hydroxybenzotriazole ester, 6-chloro-1-hydroxybenzotriazole ester, 1-hydroxy-1H-2-pyridone ester, 2-pyridylthiol ester, 2-benzothiazolylthiol ester, and S-1,3 -benzothiazol-2-yl(2Z)-(5-amino-1,2,4-thiadiazol-3-yl)(ethoxyimino)ethanethioate.

The term “salt-forming agent” in the present invention refers to an acid or a base that is capable of forming a salt with ceftaroline. A salt-forming agent includes, for example, an inorganic acid, an inorganic base, an organic acid, and an organic base. Examples of a salt-forming agent include sodium bicarbonate, sodium carbonate, potassium carbonate, potassium bicarbonate, calcium chloride, calcium carbonate, calcium acetate, ethanolamine, diethanolamine, N-methylglucosamine, hydrochloric acid, hydrobromic acid, sulfuric acid, nitric acid, phosphoric acid, p-toluenesulfonic acid, methanesulfonic acid, formic acid, trifluoroacetic acid, maleic acid, lysine, arginine, ornithine, and histidine.

In an embodiment, the salt-forming agent may be hydrochloric acid, hydrobromic acid, sulfuric acid, sodium bicarbonate, potassium bicarbonate, diethanolamine, or methanesulfonic acid.

The term “about” in the present invention, when used along with values assigned to certain measurements and parameters, means a variation of 10% from such values, or in the case of a range of values, means a 10% variation from both the lower and upper limits of such ranges.

XRPD of the sample was determined by using Panalytical X'Pert Pro X-Ray Powder Diffractometer in the range 3 to 40 degree 2 theta with a step size of 0.02 and under tube voltage and current of 45 Kv and 40 mA respectively. Copper radiation of wavelength 1.54 angstrom and Xceletor detector were used.

While the present invention has been described in terms of its specific embodiments, certain modifications and equivalents will be apparent to those skilled in the art and are intended to be included within the scope of the present invention.

### EXAMPLES

### Example 1

### Preparation of S-1,3-Benzothiazol-2-Y1(2z)-(5-Amino-1,2,4-Thiadiazol-3-Y1)(Ethoxyimino)Ethanethioate (Reactive Derivative of Formula IV)

Triphenylphosphine (16 g, 0.06 moles) and (2Z)-(5-amino-1,2,4-thiadiazol-3-yl)(ethoxyimino)ethanoic acid (Formula IV; 10 g, 0.046 moles) were treated in dichloromethane (60 mL) at 25° C. to 30° C. The resulting reaction mass was stirred for 1 to 2 hours at 25° C. to 30° C. and then cooled to 0° C. 2,2′-Disulfanediylbis(1,3-benzothiazole) (20.2 g, 0.06 moles) was added to the reaction mass at 0° C. to 5° C. Triethylamine (5.14 g, 0.051 moles) was added drop wise to the reaction mass over 5 to 10 minutes at 0° C. to 5° C. The resulting reaction mass was stirred for 3 to 4 hours at 0° C. to 5° C. and was allowed to stand for 1 to 4 hours. The solid obtained was filtered, washed with dichloromethane, and dried in an air oven for 10 to 12 hours at 40° C. to 45° C. Yield=22.2 g (88% yield)

### Example 2

### Preparation of Ceftaroline Dihydrochloride (Formula IIa)

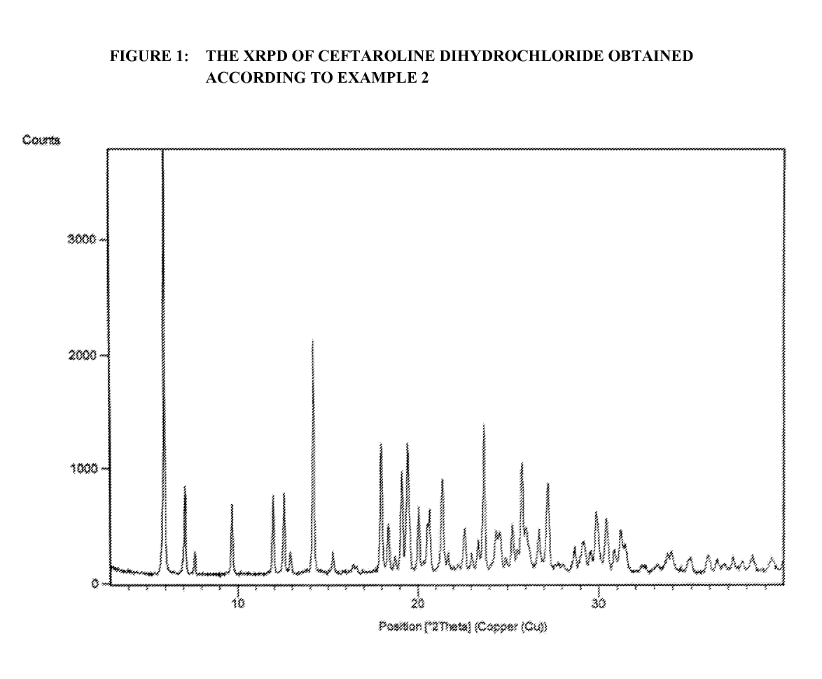

7-Amino-3-{[4-(1-methyl pyridinium-4-yl)-1,3-thiazol-2-yl]sulfanyl}-8-oxo-5-thia-1l-aza bicyclo[4.2.0]oct-2-ene-2-carboxylate hydrochloride (hydrochloride salt of Formula III; 10 g, 0.0208 moles) and S-1,3-benzothiazol-2-yl(2Z)-(5-amino-1,2,4-thiadiazol-3-yl)(ethoxyimino)ethanethioate (reactive derivative of Formula IV; 22.2 g, 0.042 moles) were treated in a mixture of tetrahydrofuran and water (3:2; 200 mL) at 25° C. to 30° C. Tributylamine (10.7 g, 0.0581 moles) was added in 10 minutes and the resulting mass was stirred for 3 to 5 hours at 30° C. to 35° C. Ethyl acetate (100 mL) was added to the resulting mass. It was stirred and the aqueous layer was separated. Ethanol (150 mL) and hydrochloric acid (35%; 30 mL) were added to the aqueous layer at 15° C. to 20° C. and the resulting mass was stirred for 10 to 12 hours at 15° C. to 20° C. The solid obtained was filtered, washed with ethanol, and dried under vacuum for 10 to 12 hours at 40° C. to 45° C. to obtain the title compound having an XRPD pattern as depicted in FIG. 1.

Yield =6.5 g (48% yield)

HPLC purity =99%

We claim:

1. A process for the preparation of a ceftaroline salt or a hydrate thereof, wherein the process comprises the steps of:
(a) treating a compound of Formula III 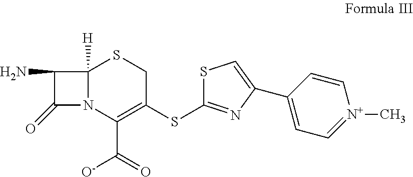 [SMILES: C[n+]1ccc(-c2csc(SC3=C(C(=O)[O-])N4C(=O)[C@@H](N)[C@H]4SC3)n2)cc1]
or an ester derivative or salt thereof with a compound of Formula IV 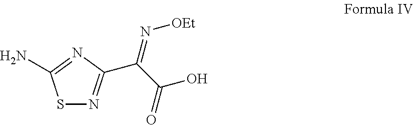 [SMILES: CCO/N=C(\C(=O)O)c1nsc(N)n1]
or a salt or reactive derivative thereof to obtain a reaction mass; and
(b) treating the reaction mass with a salt-forming agent to obtain a ceftaroline salt or a hydrate thereof.

2. The process according to claim 1, wherein the compound of Formula IV is a free acid or a salt, and a condensing agent is used.

3. The process according to claim 2, wherein the condensing agent is N,N-dicyclohexyl carbodiimide

4. The process according to claim 1, wherein a salt of the compound of Formula III is treated with a reactive derivative of the compound of Formula IV.

5. The process according to claim 4, wherein the salt of the compound of Formula III is treated with the reactive derivative of the compound of Formula IV in a solvent in the presence of a base.

6. The process according to any one of claims 1 to 5, wherein the salt of the compound of Formula III is a hydrochloride salt.

7. The process according to any one of claims 1 to 5, wherein the reactive derivative of the compound of Formula IV is S-1,3-benzothiazol-2-yl(2Z)-(5-amino-1,2,4-thiadiazol-3 -yl)(ethoxyimino)ethanethioate.

8. The process according to claim 1, wherein the salt-forming agent is hydrochloric acid, hydrobromic acid, sulfuric acid, sodium bicarbonate, potassium bicarbonate, diethanolamine, or methanesulfonic acid.

9. The process according to claim 1, wherein the ceftaroline salt is a ceftaroline dihydrochloride salt.

10. The process according to claim 5, wherein the solvent is dioxane, tetrahydrofuran, dimethylformamide, dimethylacetamide, acetone, acetonitrile, dimethylasulfoxide, water, or a mixture thereof.

11. The process according to claim 5, wherein the base is tributylamine, triethylamine, sodium carbonate, potassium carbonate, calcium carbonate, or a mixture thereof.

12. The process according to claim 1, wherein a water-immiscible solvent is added to the reaction mass formed in step a).

13. The process according to claim 12, wherein the water-immiscible solvent is ethyl acetate, isopropyl acetate, n-propyl acetate, n-butyl acetate, isobutyl acetate, amyl acetate, methylene chloride, chloroform, carbon tetrachloride, amyl chloride, cyclohexane, methyl cyclohexane, isopropyl ether, diethyl carbonate, methyl isobutyl ketone, diisopropyl ketone, diisobutyl ketone, cyclohexanone benzene, toluene, xylene, or mixtures thereof.
